(19) United States
(12) Plant Patent Application Publication
(10) Pub. No.: US 2005/0251886 P1
(43) Pub. Date: Nov. 10, 2005

(54) Strawberry plant named 'Orleans'

(50) FRAGARIA ANANASSA; STRAWBERRY

(76) Inventor: Shahrokh Khanidazeh, Baie D'Urfe (CA)

Correspondence Address: MANELLI DENISON & SELTER, 2000 M STREET NW SUITE 700, WASHINGTON, DC, 20036-3307, US

(21) Appl. No.: 10/839,287

(22) Filed: May 6, 2004

(51) Int. Cl. A01H 5/00

(52) U.S. Cl. PLT/208

(57) ABSTRACT

A new and distinct June-bearing strawberry cultivar named ‘Orléans’ is primarily adapted to the growing conditions of Eastern Central Canada and shows resistance to soil born diseases. Its upright growing habit, its small flowers with as-long-as-broad petals, its firm light-red fruit with same-sized calyx, its fruit sweetness, long shelf life and high levels of antioxidants essentially characterize ‘Orléans’.

### FIELD OF THE INVENTION

The present invention relates to a new and distinct June bearing strawberry cultivar designated as ‘Orléans’. This cultivar belongs to the genus Fragaria (×ananassa Dutch.), whose fruit are juicy, edible and usually red, and is cultivated for culinary purposes.

### BACKGROUND OF THE INVENTION

The new cultivar, tested as FIO9623-55, is the progeny of a cross made in 1996 by Shahrokh Khanizadeh between ‘L'Acadie’ and ‘Joliette’ (U.S. Plant Pat. No. 10,460). ‘L'Acadie’ is a June bearing strawberry cultivar (Fragaria×ananassa Duch.) bred for Eastern Central Canada and more specifically for Quebec growing conditions. ‘L'Acadie’ is noted for large, firm fruit, moderate resistance to leaf diseases, partial resistance to red stele (Phytophthora fragariae Hickman), and keeping quality of several days after picking or maturity in the field. ‘Joliette’ has high yields of large, moderately firm fruit and is resistant to leaf spot (Mycosphaerella fragariae Tul.) and to six North American eastern (NAE) races of red stele (Phytophthora fragariae Hickman).

The ‘Orléans’ strawberry was asexually propagated by runners at the Agriculture and Agri-Food Canada sub-station in L'Acadie, Québec and extensively tested at the same location (where it has been tested since 1997). It was reselected by Les Fraises de L'\hat{l}le d'Orléans Inc. in St Laurent, Île d'Orléans, Québec, Canada in 1999. The new variety was then further evaluated from 1999 to 2001 in controlled semi-commercial sites by our private partners, Meiosis Ltd (Kent, UK). It is presently evaluated in other provinces of Canada, in the United States, and in Europe. Clones of the claimed plant are identical to the original plant. ‘Orléans’ is now an established and stable cultivar.

### BRIEF SUMMARY OF THE INVENTION

‘Orléans’ is primarily adapted to the climate and growing conditions of Eastern Central Canada and more specifically for I'Île d'Orléans, Québec. It can tolerate low winter temperatures and shows resistance to soil born diseases. Its upright growing habit, its small flowers with as-long-as-broad petals, its firm light-red fruit with same-sized calyx its and fruit sweetness essentially characterize ‘Orléans’, as compared to ‘Kent’, ‘L'Acadie’ and ‘Joliette’. ‘Orléans’ out-yields ‘Kent’ and produces larger fruits that ripen 4-5 days after ‘Kent’ fruits. ‘Orléans’ also continues to produce fruits 4-5 days after ‘Kent’, therefore it is considered as a mid-season late cultivar. ‘Orléans’ has a much longer shelf life than varieties like ‘Chambly’ (U.S. Plant Pat. No. 8,853), ‘Kent’, ‘Annapolis’ and ‘Saint-Pierre’ and shows higher levels of antioxidants (Gallic acid, Protocatecuic acid, Catechin, P-hydroxybenzoic acid, Epicatechin, and Ellagic acid) than ‘Kent’.

### BRIEF DESCRIPTION OF THE DRAWINGS

The accompanying color photographs show typical specimens of the new variety at various stages of development as nearly true as it is possible to make in color reproductions.

FIG. 1 shows typical ‘Orléans’ field plants;

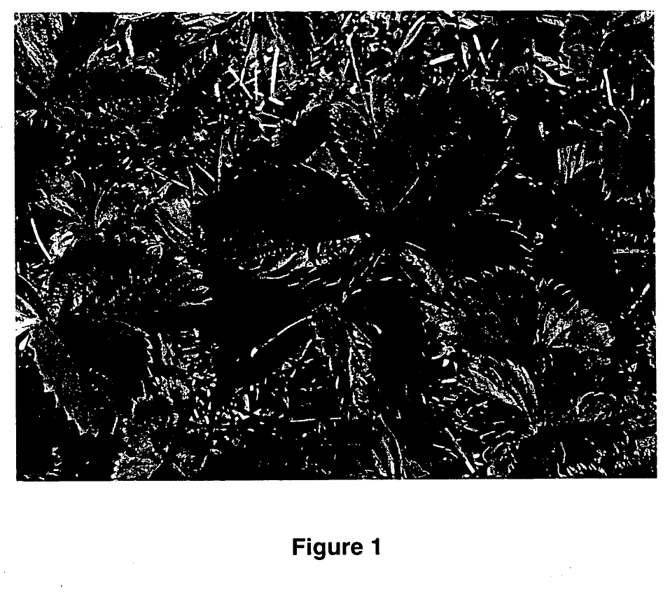

FIG. 2 shows a close-up view of a typical mature trifoliate of ‘Orléans’;

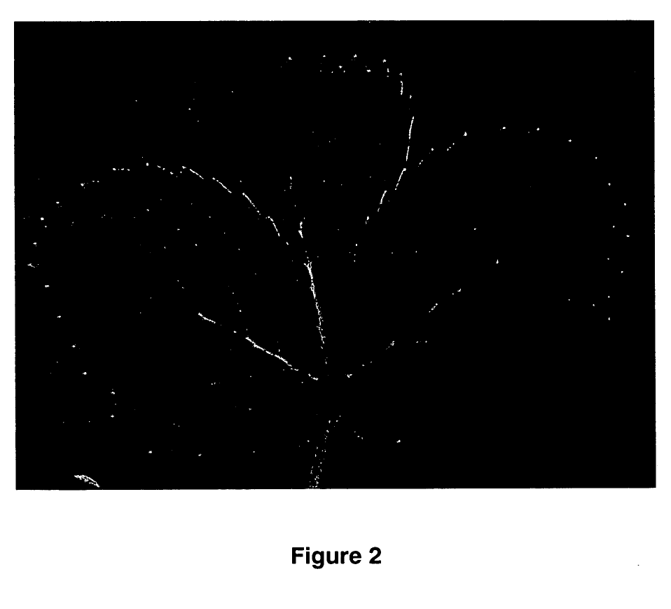

FIG. 3 shows a close-up view of typical ‘Orléans’ inflorescences;

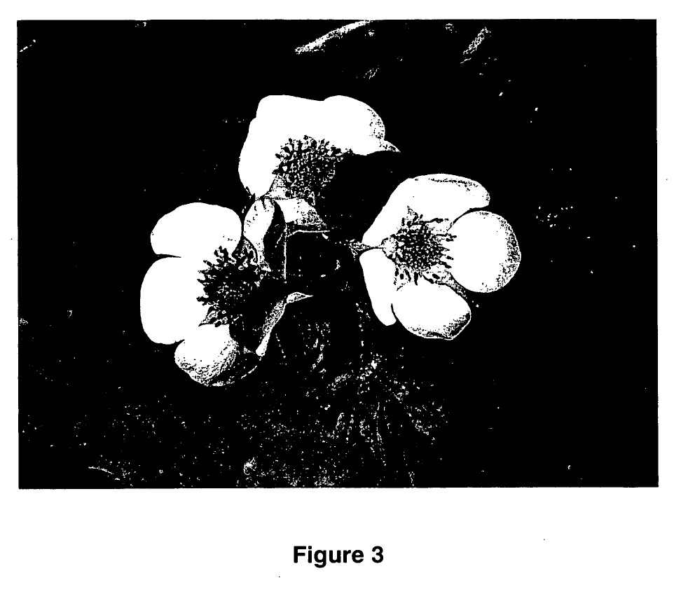

FIG. 4 shows a close-up view of typical mature and immature ‘Orléans’ field fruits, taken on Jul. 14, 2003;

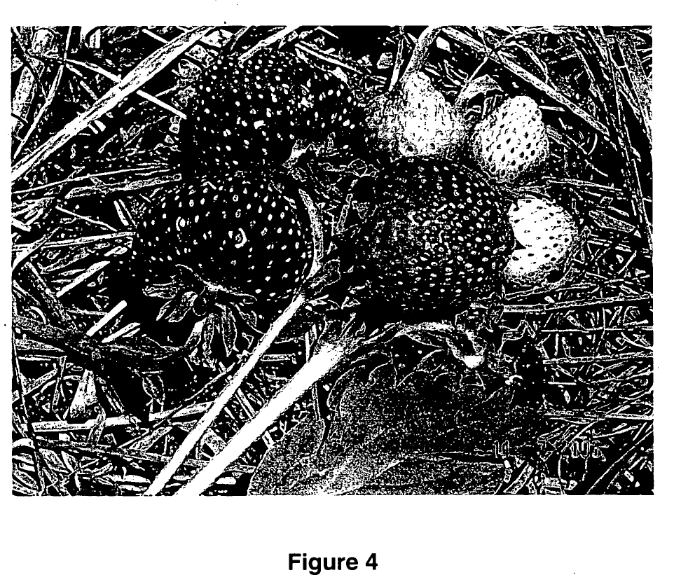

FIG. 5 shows a close-up view of ‘Orléans’ fruits;

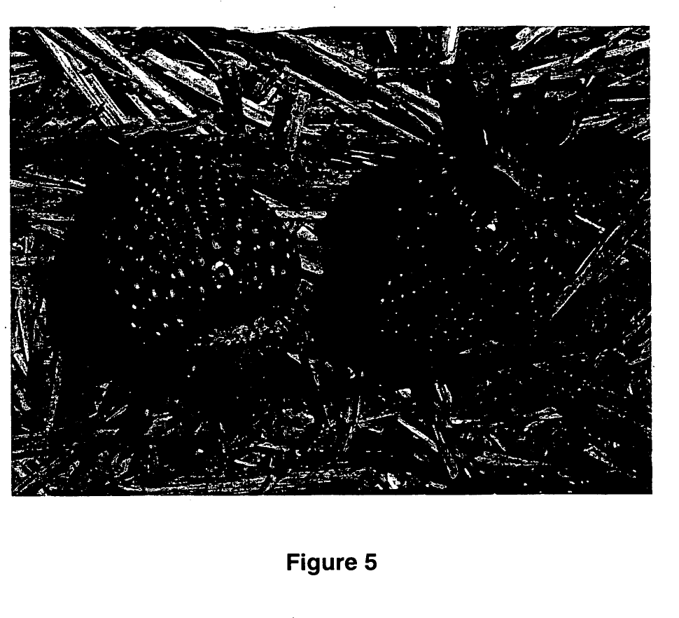

FIG. 6 shows typical internal and external fruit characteristics of ‘Orléans’;

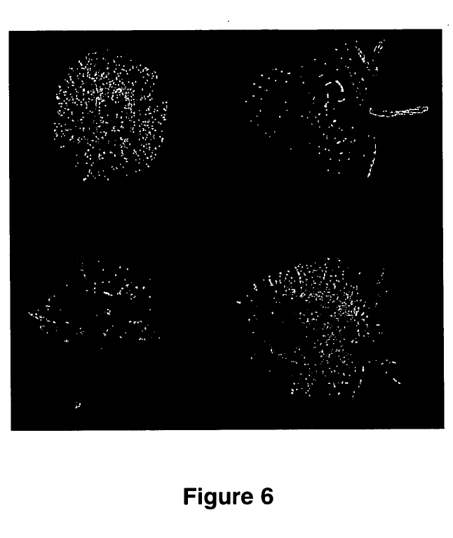

FIG. 7 shows a comparison of internal fruit characteristics between ‘Orléans’ and ‘Kent’; and

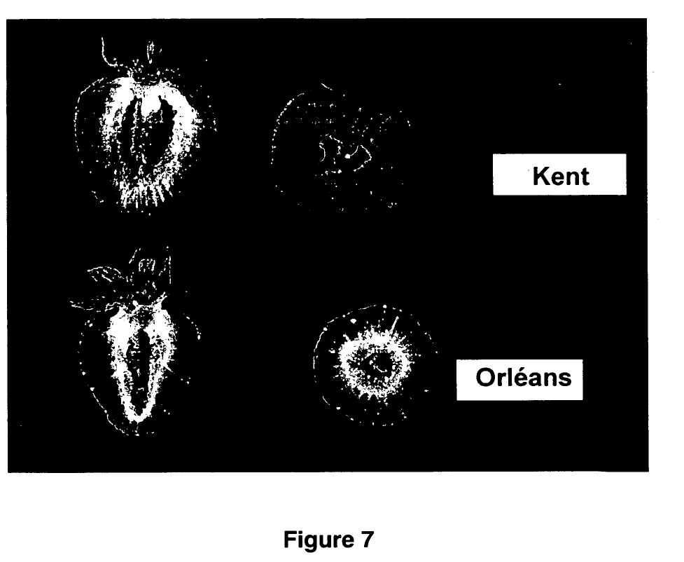

FIG. 8 shows a comparison of trifoliates between ‘Orléans’ and ‘Kent’.

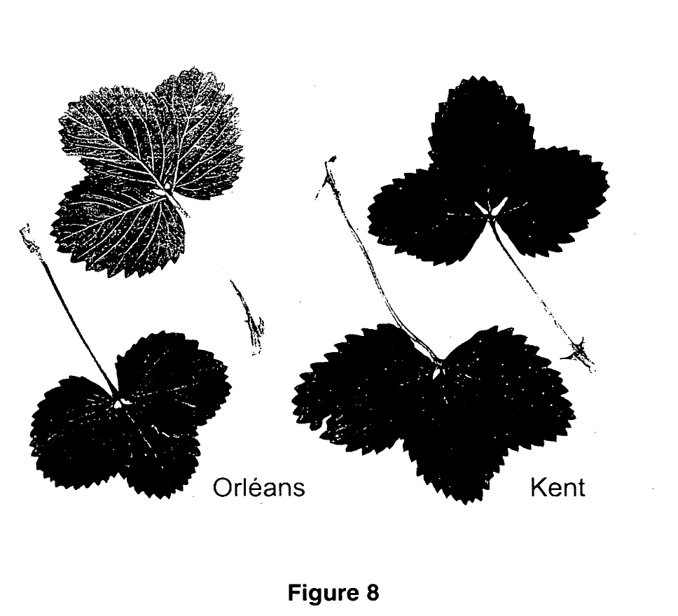

### DETAILED BOTANICAL DESCRIPTION

‘Orléans’ is a June bearing strawberry cultivar (Fragaria×ananassa Duch.) through a matching investment initiative between Agriculture and Agri-Food Canada, Les Fraises de I'\hat{l}le d'Orléans Inc. and Mc Gill University. It is a progeny (FIO9623-55) resulting from a cross between two recent released from the Horticulture Research and Development Center (HRDC) of Agriculture and Agri-Food Canada, ‘L'Acadie’ and ‘Joliette’ (U.S. Plant Pat. No. 10,460). ‘Orléans’ has high yields of very large, firm, light-red colored fruit and performs a much longer storage life than the standard variety Kent, used by many growers. It also shows higher levels of antioxidants (Gallic acid, Protocatecuic acid, Catechin, P-hydroxybenzoic acid, Epicatechin, and Ellagic acid) than ‘Kent’, which makes it ideal for growers who need to store the fruits for several days or ship them to other provinces for marketing.

The name ‘Orléans’ refers to a region east of Quebec City. This area is still recognized today as the capital for strawberry production in Quebec. L'ĺle d'Orlèans, which was once referred to as “I'ĺle nourriciére” is the oldest seigneury of New France and has since then remained known as a horticultural growing region.

Plants of ‘Orléans’ are vigorous, have an upright growing habit and produce 3 to 4 inflorescences per crown. They can tolerate winter temperatures below −30° C. (with 10 cm straw mulch cover), they perform very well on fumigated or non-fumigated soils and show resistance to soil born diseases.

### Plant Characteristics

Plant:
   Habit.—Upright.
   Density (of individual plants in hill culture or plants/m2 for matted rows).—Dense.
   Vigor.—Medium.
   Low temperature tolerance.—High.

### Stolon Characteristics

Stolon:
   Number.—Many.
   Anthocyanin coloration.—Medium to strong.
   Thickness.—Medium.
   Pubescence.—Medium to dense.

### Fruit Characteristics

‘Orléans’ fruits, fruit production and fruit quality characteristics.

TABLE 1
Yield distribution (g.m⁻¹ of row), total yield, fruit weight, ripening season,
index of crop concentration and earliness of ‘Orléans’ as compared to
other genotypes
Data collected in L'Acadie site (Québec)
Harvest - Yield (g.m⁻¹)

| Genotypes | 1^{z} | 2 | 3 | 4 | 5 | 6 |
|---|---|---|---|---|---|---|
| Orléans | 0.0 | 0.0 | 755.5 | 1733.1 | 2131.7 | 2549.1 |
| Annapolis | 13234 | 2439.8 | 3253.9 | 1480.6 | 771.4 | 1368.5 |
| Chambly | 3137.7 | 1342.8 | 1069.7 | 563.7 | 0.0 | 0.0 |
| Honeoye | 1568.6 | 1800.0 | 2530.3 | 1884.9 | 641.3 | 256.6 |
| Kent | 324.6 | 1246.2 | 1593.7 | 1270.5 | 1587.8 | 985.7 |
| Mira | 332.2 | 821.8 | 2287.0 | 2316.9 | 1729.5 | 1784.6 |
| St-Pierre | 0.0 | 0.0 | 0.0 | 1643.7 | 1506.9 | 0.0 |

| Harvest - Yield (g.m⁻¹) | | | | | | | |
|---|---|---|---|---|---|---|---|
| | | | | | Total | Wt./ | Ripen- |
| | | | | | yield | fruit | ing |
| Genotypes | 7 | 8 | 9 | 10 | (g.m⁻¹) | (g)^{y} | season^{x} |
| Orléans | 2075.4 | 1415.8 | 1399.4 | 289.5 | 12349.6 | 12.6 | M |
| Annapolis | 0.0 | 40.6 | 0.0 | 0.0 | 10683.3 | 10.7 | E |
| Chambly | 0.0 | 0.0 | 0.0 | 0.0 | 6113.8 | 10.4 | EM |
| Honeoye | 203.7 | 13.4 | 0.0 | 0.0 | 8898.7 | 9.5 | EM |
| Kent | 646.9 | 304.5 | 240.1 | 203.6 | 8403.6 | 8.2 | EM |
| Mira | 1074.3 | 25.7 | 0.0 | 0.0 | 10372.1 | 10.7 | LM |
| St-Pierre | 3326.4 | 1371.1 | 0.0 | 687.6 | 8535.6 | 9.9 | L |

^{z}Number of times fruits were harvested during the season LSD_{0.05}
^{y}Average over 4 years (1995-1998), minimum of 4 replications per year, data from the l'Acadie site.
^{x}L = Late,
LM = Late-Midseason,
M = Midseason,
EM = Early-Midseason.

TABLE 2
Firmness, flavour, skin color, leaf disease susceptibility and shelf life of
“Orléans, as compared to other genotypes^{z} Data
collected in L'Ile d'Orléans (St Laurent, Québec) site.

| | | | | Leaf disease^{y} | |
|---|---|---|---|---|---|
| Genotypes | Firmness^{y} | Flavor^{y} | Skin color^{y} | susceptibility | Shelf life^{x} |
| Orléans | 4.4 | 4.5 | 2.3 | 4.8 | 5.0 |
| Annapolis | 2.9 | 2.0 | 2.0 | 3.4 | 2.0 |
| Honeoye | 3.0 | 2.9 | 4.1 | 3.9 | 1.5 |
| Kent | 2.8 | 4.0 | 3.4 | 2.3 | 0.5 |
| Mira | 2.7 | 1.8 | 2.0 | 3.4 | 1.0 |
| St-Pierre | 3.3 | 3.4 | 2.2 | 3.0 | 2.5 |

^{z}Averaged over 3 years (1999-2001), minimum of four replications per year.
^{y}Data were transformed to arcsin prior to analysis of variance (SAS institute, 1988).
Firmness: 1 = very soft, 5 = very firm
Flavor: 1 = poor, 5 = excellent
Skin color: 1 = very pale, 5 = dark red
Leaf disease susceptibility: 1 = very susceptible, 5 = resistant
^{x}Number of days at room temperature for which the fruits were more than 95% marketable.

Fruit:
   Ratio of length/maximum width.—As long as broad to slightly longer than broad.
   Size.—Medium to large.
   Predominant shape.—Conical (slightly globose).
   Difference in shape between primary and secondary fruits.—Non to very slight.
   Band without achenes.—Narrow.
   Unevenness of surface.—Weak.
   Skin color.—Light red.
   Evenness of color.—Slightly uneven.
   Glossiness.—Strong.
   Insertion of achenes.—Below surface.
   Insertion of calyx.—Level.
   Attitude of the calyx segments.—Clasping to reflexed.
   Size of the calyx in relation to fruit diameter.—Same size.
   Adherence of the calyx.—Strong.
   Attitude of sepals.—Reflexed.
   Firmness of flesh (when fully ripe).—Firm.
   Color of flesh.—Orange to medium red.
   Evenness of color of flesh.—Even.
   Sweetness.—Medium to strong.
   Texture when tested.—Fine.
   Acidity.—Weak.
   Time of flowering (50% of plants at first flower).—Medium.
   Harvest maturity (50% of plants with ripe fruits).—Mid-season.
   Type of bearing.—Not everbearing.

‘Orléans’ differs from its parents (‘Joliette’ and ‘L'Acadie’) in terms of fruit shape, calyx and fruit color. As stated earlier, ‘Orléans’ fruit is globose-conic with reflexed sepals rested on a white short neck with very light glossy red color, whereas ‘Joliette’ fruit change from globose to short-wedge shape during the harvest. ‘Joliette’ skin is reddish and its sepals are not reflexed. ‘L'Acadie’ fruits are shiny pale red, with a necked-conic predominant shape and the calyces are semi-reflexed.

Chemical analysis of the ‘Orléans’ fruits reveal high levels of free epicatechin and ellagic acid and above the average to very high levels of bound catechin, epicatechin and ellagic acid. Free antioxidants are immediately available to the plant and therefore help its resistance against diseases or other external stresses. They also act to extend shelf life and enhance quality preservation by delaying senescence created by oxidative degradation. Bound antioxidants, which are measured after hydrolyzing samples, are chemicals that can provide a health benefit after ingestion. For example, ellagic acid and catechin have been shown to have anti-carcinogenic and anti-inflammatory properties (Ellagic acid, an anticarcinogen in fruits, especially in strawberries: a review. Maas et al., HortScience 26 :10-14, 1991).

The ‘Orléans’ fruit has a long shelf life, over 5 days at 4° C. (Table 2) and 3-4 days at room temperature.

### Foliage Characteristics

Leaf:
   Green color of upper side.—Light to medium light.
   Profile (angle terminal leaflet subtends to the petiole).—Slightly concave.
   Blistering (interveinal blisters).—Weak.
   Number of leaflets.—Three.
   Leaflet number of serrations.—24-30.
Terminal leaflet:
   Profile.—Flat to slightly cupped.
   Length/width ratio.—As long as broad.
   Shape of base.—Obtuse to rounded.
   Shape of teeth.—Acute to obtuse.
Petiole:
   Length.—0-15 cm.
   Pubescence.—Medium.
   Pose of hairs.—Upwards to outwards.

### Flowers and Inflorescences Characteristics

Inflorescence:
   Position relative to foliage.—Below to level with.
   Attitude of fruiting trusses (at first picking).—Semi erect.
   Length of fruiting trusses.—Short to medium.
Flowers:
   Size.—Small.
   Diameter of calyx relative to corolla.—Larger.
   Diameter of inner calyx relative to outer (on secondary flowers).—Same size to larger.
   Spacing of petals (secondary flowers with 5 to 6 petals).—Touching to touching-overlapping.
   Petal length/width ratio (on secondary flowers).—As long as broad.

Some symptoms of powdery mildew were noted on ‘Orléans’ plants, as observations began in 1996 during prolonged high humidity. However, ‘Orléans’ plants are less susceptible to mildew than ‘Kent’. ‘Orléans’ plants are resistant to leaf scorch (Diplocarpon earlina Ell. & Ev.), leaf blight (Dendrophoma obscurans Ell. & Ev.) and leaf spot (Mycosphaerella fragariae (Tul.) Lindau), as compared to ‘Kent’ control plants that are very susceptible to all these leaf diseases. ‘Orléans’ is resistant to soil-born diseases.

‘Orléans’ plants are more vigorous than ‘Chandler’ in both fumigated and non-fumigated soils. ‘Orléans’ is recommended for Eastern Central Canada, especially in areas where the climate is similar to that in the strawberry production areas of Québec, for example, I'Acadie (35 km South East of Montreal Island, Québec, lat. 45°N and 46 m elevation). The climate at L'Acadie, where ‘Orléans’ has been extensively tested, is characterized by extreme low temperatures in winter (<−25° C.); cool, wet, humid conditions in spring; and warm, humid conditions in summer (25-35° C., 70% RH). It has a clay loam soil with moderate to low drainage and little snow cover during the winter.

1. A new and distinct strawberry plant named ‘Orléans’ as described and illustrated herein.
